(19) United States
(12) Patent Application Publication
(10) Pub. No.: US 2004/0133938 A1
(43) Pub. Date: Jul. 8, 2004

(54) Use of lipoic acid in plant culture media

(76) Inventors: Yinghui Dan, Raleigh, NC (US); Tichafa R. I. Munyikawa, Ballwin, MO (US); Kimberly A. Rayford, St. Louis, MO (US); Caius M. T. Rommens, Boise, ID (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/715,910

(22) Filed: Nov. 18, 2003

Related U.S. Application Data
(60) Provisional application No. 60/427,437, filed on Nov. 19, 2002.

(51) Int. Cl. C12N 15/82; A01H 1/00; A01N 43/26

(52) U.S. Cl. 800/278; 504/290

(57) ABSTRACT

Plant transformation media containing lipoic acid has been found to improve the efficiency of transformation or regeneration. Methods of using lipoic acid in the transformation of tomato, potato, soybean, wheat, and cotton are particularly described.

This application claims priority to U.S. Provisional Application 60/427,437, filed Nov. 19, 2002, herein incorporated by reference in its entirety.

### FIELD OF THE INVENTION

This invention relates to plant tissue culture media designed to more efficiently obtain transgenic plant cells and regenerated plants therefrom, and more particularly to plant transformation media containing an effective amount of lipoic acid or an analog thereof.

### BACKGROUND OF THE INVENTION

The production of transgenic plants requires that the plant cells or tissue be transformed with a gene of interest and then regenerated into a whole plant. The most effective current methods for transforming plants to introduce a gene of interest require that the cells or tissue be maintained in plant culture media for several weeks to effect selection or sufficient growth for the next step. Many plants, particularly commercially important ones, are difficult to maintain in tissue culture, and this poses a limitation on the number of transgenic plants that can successfully be regenerated from culture. One reason posed to explain the difficulty of plant tissue in culture media is that the plant tissue/cells are stressed when forced to grow in tissue culture media. Potential reasons for this may include production of free radicals or reactive oxygen species, which damage cells, or activation or alteration of metabolic pathways.

Oxidative stress in culture can play an important role (Bensen, In Vitro Cell. Dev. Biol.-Plant, 36:163-170, 2000). Antioxidants such as ascorbic acid, alpha-tocopherol, 3,5-dibutyl-4-hydroxytoluene (BHT), cysteine, silver nitrate, polyvinylpolypyrrolidone (PVP), dithiothreitol, phenoxane, citrate, glutathione, phytic acid, nordihydroguaiaretic acid (NDGA), and activated charcoal (all usually in millimolar amounts) have been used in culture to reduce effects from oxidation.

Thus, there is a continuing need to provide plant transformation media that improve the ability of the transformed tissue/cells to survive in the media during the transformation/regeneration process so as to increase the efficiency of the overall transformation process. In this regard, lipoic acid (6,8-thioctic acid or 1,2-dithiolane-3-pentanoic acid) is a sulfur-containing compound involved in several multienzyme complexes such as pyruvate dehydrogenase, α-ketoglutarate dehydrogenase, branched-chain keto acid dehydrogenase, and glycine decarboxylase complex. Free lipoic acid and dihydrolipoic acid are also metabolic antioxidants that are able to scavenge the reactive oxygen species, to recycle other antioxidants (vitamin C, glutathione, and vitamin E), and even to intervene in redox regulation of gene transcription (for a review see Packer et al., Free Radical Biol. Med. 19:227-250, 1995). Other active analogs of lipoic acid have been identified (see, for example, U.S. Pat. No. 6,090,842, U.S. Pat. No. 5,334,612, U.S. Pat. No. 2,933,430, and U.S. Pat. No. 5,508,275, hereby incorporated by reference hereto).

### SUMMARY OF THE INVENTION

A method for genetically transforming a plant cell, tissue or other suitable explant and regenerating a transformed plant therefrom is provided. In accordance with the present invention, the method provides for introducing a nucleic acid into the genome of a plant cell wherein an effective amount of lipoic acid or a lipoic acid analog is included in the transformation media. In the practice of the method, the plant cell, tissue or explant is placed in contact with a transformation media comprising an amount of lipoic acid or a lipoic acid analog sufficient to enhance the efficiency of transformation and/or regeneration, and/or the survivability of the plant cell, tissue or explant during the transformation and/or regeneration process as compared to a plant cell, tissue or explant being transformed in the absence of lipoic acid or an analog thereof.

The invention further provides plant transformation media compositions comprising an effective amount of lipoic acid or a lipoic acid analog. The media may be liquid, solid or semi-solid, and the lipoic acid or analog thereof may be included in any of the particular media used during the transformation process, e.g., the inoculation, co-cultivation, delay, selection, shoot induction, elongation, regeneration or rooting media.

Also provided is a method of improving the efficiency of transformation and/or regeneration of a plant cell, tissue or explant in the presence of lipoic acid or an analog thereof, wherein the lipoic acid or analog thereof is included in the plant transformation media in which the plant cells, tissue or explant are cultured during the transformation process.

More specifically, the invention also provides a method for transforming dicotyledonous and monocotyledonous plant tissue and regenerating fertile transgenic plants therefrom comprising the inclusion of an effective amount of lipoic acid or an analog thereof in at least one of the plant transformation media during the transformation and/or regeneration process.

### BRIEF DESCRIPTION OF THE DRAWINGS

FIG. 1 is a schematic map of plasmid pMON 15715.

FIG. 2 is a schematic map of plasmid pMON 17270.

FIG. 3 is a schematic map of plasmid pMON 15737.

### DETAILED DESCRIPTION OF PREFERRED EMBODIMENTS

It has been discovered that the inclusion of lipoic acid or a lipoic acid analog in at least one plant transformation media during the transformation and/or regeneration process increases the efficiency of transformation of a plant explant with a selected nucleic acid fragment and/or the regeneration of a transgenic plant therefrom and the survivability of plant cells, tissue or other explant during the transformation and regeneration process. The inclusion of lipoic acid has proved useful as an addition to media used for monocotyledonous and dicotyledonous plants at various steps throughout the transformation and regeneration process.

“Transformation media” or “plant transformation media” as used herein, refers to the plant tissue culture media, whether liquid, solid or semi-solid, used during the process of the transformation of plant cells, tissues, parts or other plant tissue explants and subsequent regeneration of whole, transgenic plants therefrom. Depending upon the plant species being transformed and the transformation process being used, the transformation media may include, but is not limited to, the isolation media, induction media, delay media, selection media and/or regeneration media.

“Efficiency of transformation or regeneration” or “transformation efficiency,” as used herein, refers to the percentage of transgenic events produced per explant or the percentage of transgenic plants produced per explant. The efficiency of transformation may also be described in the number of “escapes” resulting from the transformation process.

“Survivability” of a plant cell, tissue, part or other explant during the transformation and regeneration process, as used herein, refers to the ability of the cell, tissue, part or other explant to flourish in the transformation media with little or no browning or other disadvantageous characteristics to limit its ability to continue to divide and grow in the media.

An “event,” as used herein, refers to a particular genomic insertion of the desired gene into a specific plant.

An “escape,” as used herein, refers to a plant that survives the selection process without having the gene encoding for resistance to the selectable marker stably transformed into the plant genome.

A “plant stress condition,” as used herein, refers to less than optimal conditions necessary for maintaining healthy growth or maintenance of plant cells or tissue in plant transformation media, such as by repeated media transfers, limiting nutrients (including water and light), less than optimal quality of plant tissue or cells such as by wounding or excessive handling. This list is not intended to be exclusive of other stress conditions known to those skilled in the art of plant transformation.

In a preferred embodiment of the invention and as further detailed in the Examples below, lipoic acid has been added to plant transformation media at various steps of the transformation process in at least 5 different plant species to optimize its use for the particular plant species. Although the same effect was not seen for each plant species and its particular transformation process, an overall positive result was seen for each in terms of improving plant transformation efficiency. Although not intending to be bound hereby, the inclusion of lipoic acid in a plant transformation media seems to be most beneficial during the stages of transformation and/or regeneration where the plant tissues are exposed to plant stress conditions.

The amount of lipoic acid to include in the plant transformation media and in which media during the transformation/regeneration process it should be included to be most efficacious varies from plant species to plant species and the transformation system being employed. For example, in an Agrobacterium-based MicroTom (tomato) transformation process, lipoic acid has been benefically included in the shoot induction media, in concentrations from about 5-100 μM, about 5-50 μM and about 10 μM, to be most effective. An Agrobacterium-based soybean transformation process, however, provides benefits at higher concentrations of lipoic acid, from about 100-2000 μM, and about 200-1500 μM, and about 250 μM, and is most effective when included in the co-culture media. In a potato Agrobacterium transformation system, potato explants respond well at lipoic acid concentrations from about 6 μM to about 100 μM, and about 6 μM to about 50 μM, and about 8-10 μM in the delay media (between co-culture and selection). In the monocotyledonous wheat plant using an Agrobacterium-based transformation system, wheat tissue responds best in a lipoic acid concentration of about 25-50 μM in the delay, selection, and first regeneration media. The inclusion of lipoic acid in the selection media for a cotton Agrobacterium-based transformation system increases the frequency of embryogenic calli at concentration from about 50-100 μM.

In accordance with the invention, lipoic acid analogs that provide the same function that lipoic acid does when used in accordance with this invention could also be used in this invention. U.S. Pat. No. 6,090,842, herein incorporated by reference in its entirety, discloses several analogs that are stable in biological systems. U.S. Pat. No. 5,508,275, herein incorporated by reference in its entirety, discloses several analogs with lipophilic components. U.S. Pat. No. 2,933,430, herein incorporated by reference in its entirety, discloses several compounds related to lipoic acid.

Suitable analogs could include, but are not limited to, the following as well as other analogs that have similar activity and function.

Embodiments of the compounds are given according to formula I:

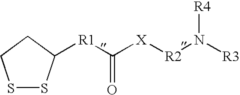

wherein

R1 and R2 independently denote a methylene, ethylene or unbranched or branched C₃₋₁₆ alkylene, alkenylene or alkynylene group that is unsubstituted or substituted with one or more halogen, hydroxyl or amine groups, wherein in said unbranched or branched C₃₋₁₆ alkylene, alkenylene or alkynylene group an internal alkylene carbon atom in the carbon backbone thereof can be replaced by an oxygen atom,

R3 and R4

(i) independently denote

(a) hydrogen,

(b) a methyl, ethyl, vinyl or unbranched or branched C₃-1₆ alkyl, alkenyl or alkynyl group that is unsubstituted or substituted with one or more halogen, hydroxyl or amine groups, wherein in said unbranched or branched C₃-1₆ alkyl, alkenyl or alkynyl group an internal alkylene carbon atom in the carbon backbone thereof can be replaced by an oxygen atom,

(c) a cycloalkyl, alkylcycloalkyl, alkenylcycloalkyl or alkynylcycloalkyl group having 5 to 16 carbon atoms that is unsubstituted or substituted with one or more halogen, hydroxyl or amine groups, or

(d) an aryl, alkaryl, aralkyl, alkenylaryl, aralkenyl, alkynylaryl or aralkynyl group having 6 to 16 carbon atoms that is unsubstituted or substituted with one or more halogen, hydroxyl or amine groups, or

(ii) jointly with the nitrogen atom form a cyclic or aromatic amine that is unsubstituted or substituted with one or more alkyl, alkenyl, alkynyl, halogen, hydroxyl or amine groups, X denotes O, S, —NH—or —NR5—, and

R5 denotes methyl, ethyl, or unbranched or branched C₃₋₁₆ alkyl,

or a pharmaceutically acceptable salt thereof, wherein said compound is in equilibrium with a protonated form thereof.

Both the R and the S enantiomer are considered to be within the scope of the present invention and specifically of formula I.

In a preferred embodiment, the compound is in equilibrium with the protonated form thereof at physiological pH.

Exemplary R1 and R2 groups include methylene, ethylene, trimethylene, tetramethylene, pentamethylene, hexamethylene, octamethylene, decamethylene, dodecamethylene, tetradecamethylene, hexadecamethylene; propylene; chloromethylene, methylmethylene; ethylethylene, propylethylene, butylethylene; 1,1-dimethylethylene, 1,2-dimethylethylene, 1-methyl-2-ethylethylene, 1,1-diethylethylene; 1-methyltrimethylene, 2-methyltrimethylene, 3-methyltrimethylene, 1,1-dimethyltrimethylene, 1-methyl-1-ethyltrimethylene, 1,2,3-trimethyltrimethylene; 1-methyltetramethylene, 2,2-dimethyltetramethylene; 2-methylhexamethylene; 4-methyloctamethylene; 2,4-dimethyldecamethylene; vinylene; 1-propylene, 2-propylene, 1-methyl-1-propylene; 1-butenylene, 2-butenylene, 3-butenylene, 1-methyl-1-butenylene, 2-ethyl-3-butenylene; 1-pentenylene, 2-pentenylene, 4-ethyl-3-pentenylene; 1,3-butadienylene, 2-methyl-1,3-butadienylene; 1,3-pentadienylene, 2,4-pentadienylene; ethynylene; 1-propynylene, 2-propynylene, 2-methyl-1-propynylene; 1-butynylene, 2-butynylene, 3-buten-1-ynylene; chloromethylene; 1-chloroethylene, 2-fluoroethylene; 2,2-dichlorotetramethylene; 1-hydroxyethylene; 2-hydroxytrimethylene, 1,2-dihydroxytrimethylene; 1-chloro-3-hydroxyhexamethylene; 1-aminoethylene; 2,4-diaminohexamethylene; 1-methyl-3-aminotetramethylene; and the like.

Additional exemplary R1 and R2 groups include groups in which at least one non-terminal carbon atom in the carbon backbone of the group is replaced by an oxygen atom. Such groups include, without limitation, —CH₂—O—CH₂—; —CH₂CH₂—O—CH₂—; —CH₂—O—CH₂CH₂—; —CH₂CH₂—O—CH₂CH₂—; —CH₂CH₂CH₂—O—CH₂CH₂—, —CH(CH₃)CH₂—O—CH₂CH₂—; —C(CH₃)₂CH₂—O—CH₂CH₂—; —CHClCH₂CH₂—O—CH₂CH₂—, —CH(OH)CH₂CH₂—O—CH₂CH₃—; —CH₂CH₂—O—CH₂CH₂—O—CH₂CH₂—; —(CH₂CH₂—O)₃—CH₂CH₂—; and the like. Such structures include ether and conjugated ether units.

If desired, R 1 or R2 can also include a substituent that is in equilibrium with a protonated form thereof at physiological pH. Such a compound will have a positive charge of at least +2. Preferably, the compound bears at most two positive charges.

Exemplary R3 and R4 groups include methyl, ethyl, propyl, butyl, pentyl, hexyl, octyl, decyl, dodecyl, tetradecyl, hexadecyl; isopropyl; isobutyl, sec-butyl, tert-butyl; isopentyl, neopentyl, tert-pentyl; isohexyl; isooctyl; vinyl; allyl, 1-propenyl, isopropenyl; 1-butenyl, 2-butenyl, 3-butenyl, 1,3-butadienyl, 2-methyl-3-butenyl; 2-pentenyl; ethynyl; 2-propynyl; 4-pentynyl; chloromethyl; 1-chloroethyl, 2-fluoroethyl; 2-chloropropyl; 1,1-difluorobutyl; 2-hydroxypropyl; 1-hydroxybutyl, 2,3-dihydroxybutyl; 2-fluoro-4-hydroxyhexyl; 2-aminopropyl; 1,3-diaminohexyl; and the like.

Additional exemplary R3 and R4 groups include groups in which at least one non-terminal (internal) alkylene carbon atom in the carbon backbone of the group is replaced by an oxygen atom. Such groups include, without limitation, —CH₂—O—CH₃; —CH₂CH₂—O—CH₃; —CH₂—O—CH₂CH₃; —CH₂CH₂—O—CH₂CH₃; —CH₂CH₂CH₂—O—CH₂CH₃, —CH(CH₃)CH₂—O—CH₂CH₃; —C(CH₃)₂CH₂—O—CH₂CH₃; —CF₂ CH₂CH₂—O—CH₂CH₃; —CH(NH₂)CH₂CH₂—O—CH₂CH₃; —CH₂CH₂—O—CH₂CH₂—O—CH₂CH₃; —(CH₂CH₂—O)₃—CH₂CH₃; and the like.

Unsubstituted and substituted cyclic R3 and R4 groups include cyclopentyl, cyclohexyl, 4-chlorocyclohexyl, 3,5-dimethylcyclohexyl, 4-tert-butylcyclohexyl, 3-cyclohexylbutyl, 4-cyclohexyl-2-butenyl, and the like. Polycyclic groups can also be used.

Unsubstituted and substituted aromatic R3 and R4 groups include phenyl, 4-chlorophenyl, 4-ethylphenyl, 3-phenylpropyl, 2-chloro-5-phenylpentyl, and the like. Polycyclic aromatic groups such as naphthyl, and partially aromatic polycyclic groups such as indyl, which can be further substituted with alkyl, alkenyl, alkynyl, halogen, hydroxyl or amine groups, can also be used.

Furthermore R3 and R4 can, together with the nitrogen atom to which they are bonded, form a cyclic or aromatic amine group, including polycyclic groups. Exemplary groups formed jointly by R3, R4 and N in formula I include: 1-pyridyl; 3-carboxy-1-pyridyl (nicotinic acid derivative); 1-imidazolyl; 1-indolyl; 1-piperidyl; 1-pyrazolyl; 1-benzpyrazolyl; and the like. The cyclic or aromatic amino groups can also be further substituted with one or more alkyl, alkenyl, alkynyl, halogen, hydroxyl or amine groups.

As with R1 and R2, the R3 and R4 groups can also include an additional substituent that is positively charged at physiological pH, resulting in a compound with a charge of at least +2. Again, it is preferable that the total charge of the inventive compound not exceed +2. More particularly, the charge of the compound is +1 at physiological pH when it is dissociated from its counterion in solution.

Preferred embodiments include those in which X=—NH—, and more specifically those in which R1 and R2 are polyalkylene groups, and R3 and R4 are methyl groups or jointly form an unsubstituted or substituted pyridine or imidazole group together with the nitrogen atom. Particularly preferred is the embodiment in which R1 is —(CH₂)₄, R2 is —(CH₂)₂—, and R3 and R4 are methyl groups. In a preferred variant, R3 and R4 are hydrogen atoms.

More generally, compounds according to the invention are provided according to formula II:

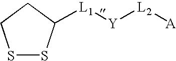

In formula II,

L₁ and L₂ independently denote (i) a methylene group or a C₆₋₁₀ arylene group that is unsubstituted or substituted with a halogen, hydroxyl, amine or unbranched or branched C₃₋₁₆ alkyl, alkenyl or alkynyl group or (ii) a linking group having a carbon backbone that includes 2 to 16 carbon atoms, wherein a carbon atom in said carbon backbone can be replaced by an oxygen atom, an unsubstituted or substituted amine group, a sulfur atom, an unsubstituted or substituted C₆₋₁₀ aryl group or a combination thereof,

Y denotes an ester, thioester, urethane or unsubstituted or alkyl-substituted amide linkage, and

A denotes a group containing a nitrogen atom that is in equilibrium with a protonated form thereof,

or a pharmaceutically acceptable salt thereof.

In formula II, A includes all groups of the form —NR3R4 as set forth above. Further exemplary A groups include imine groups having the formulae IIa-b:

—C(R7)=N—R8 IIa

—N═CR7R8 IIb

wherein

R7, R8 denote hydrogen, a methyl group, a C₆₋₁₀ aryl group that is unsubstituted or substituted with a halogen, hydroxyl, amine or unbranched or branched C₃₋₁₆ alkyl, alkenyl or alkynyl group, or an unbranched or branched C₂₋₁₆ alkyl, alkenyl or alkynyl group that is unsubstituted or substituted with a halogen, hydroxyl or amine group, and guanidine groups having the formula IIc:

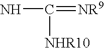

wherein R9, R10 denote hydrogen or C₁₋₆ is alkyl.

The A group can also include additional linking groups, for example additional L groups, and additional ester, thioester, urethane and/or amide linkages, affording a polymer-like structure of the form —Y-L-Y— . . . .

Exemplary L₁ and L₂ groups include those set forth above in connection with the R1 and R2 groups of formula I. Additional exemplary L₁ and L₂ groups include groups in which at least one non-terminal carbon atom in the C₂₋₁₆ carbon backbone of the group is replaced by a sulfur atom. Such groups include, without limitation, —CH₂—S—CH₂—; —CH₂CH₂—S—CH₂—; —CH₂—S—CH₂CH₂—; —CH₂CH₂—S—CH₂CH₂—; —CH₂CH₂CH₂—S—CH₂CH₂—; —CH(CH₃)CH₂—S—CH₂CH₂—; —C(CH₃)₂ CH₂—S—CH₂CH₂—; —CHClCH₂CH₂—S—CH₂CH₂—; —CH(OH)CH₂CH₂—S—CH₂CH₃—; —CH₂CH₂—S—CH₂CH₂—S—CH₂CH₂—; —(CH₂CH₂—S)₃—CH₂CH₂—; and the like.

Further exemplary R1 and R2 groups include groups in which at least one carbon atom in the C₂₋₁₆ carbon backbone of the group is replaced by an unsubstituted or substituted amine group. In the latter case, the amine group preferably is substituted with an unbranched or branched C₃₋₁₆ alkyl, alkenyl or alkynyl group. Such groups include, without limitation, —CH₂—NH—CH₂—; —CH₂—N(CH₃)—CH₂—; —CH₂—N(CH₂CH₃)—CH₂—; —CH₂CH₂—NH—CH₂—; —CH₂—N(CH₂CH₃)—CH₂CH₂—; —CH₂CH₂—NH—CH₂CH₂—; —CH₂CH₂CH₂—N(CH₂CH₂CH₃)—C₂ CH₂—; —CH(CH₃)CH₂—NH—CH₂CH₂—; —C(CH₃)₂ CH₂—NH—CH₂CH₂—; —CH₂CH₂CH₂—N(CH₂CH₂CH₃)—CH₂CH₂—; and the like.

Additional L₁ and L₂ groups include unsubstituted or substituted C₆₋₁₀ arylene groups, and groups in which one or more carbon atoms in the C₂₋₁₆ carbon backbone thereof are replaced with an unsubstituted or substituted aryl group. Combinations of aryl and oxygen, sulfur and/or amine groups can also be included, as well as partially aromatic polycyclic groups. Polymeric structures including repeated aryl, alkaryl, etc. units can also be included.

Exemplary L₁ and L₂ groups include, without limitation,

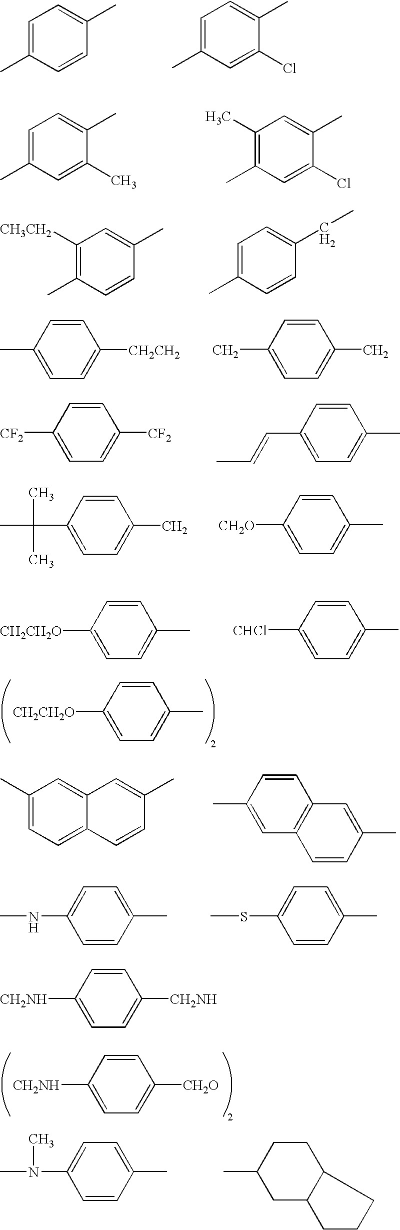

and the like.

Still more generally, the invention provides compounds according to the formula III:

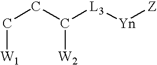

In formula III,

W₁, W₂ (i) jointly denote—S—S—, —S(O)—S—or —S—S(O)—, or (ii) individually denote a group including an oxidized sulfur atom,

L₃ denotes (i) a single bond, (ii) a methylene group or a C₆₋₁₀ arylene group that is unsubstituted or substituted with a halogen, hydroxyl, amine or unbranched or branched C₃-1₆ alkyl, alkenyl or alkynyl group or (iii) a linking group having a carbon backbone that includes 2 to 16 carbon atoms, wherein a carbon atom in said carbon backbone can be replaced by an oxygen atom, an unsubstituted or substituted amine group, a sulfur atom, an unsubstituted or substituted C₆₋₁₀ aryl group or a combination thereof,

Y denotes an ester, thioester, urethane or unsubstituted or substituted amide linkage,

n denotes 0 or 1, and

Z denotes a group that is in equilibrium at physiological pH with a protonated form thereof and increases the intracellular retention time of said compound with respect to the analogous compound having a carboxyl group,

or a pharmaceutically acceptable salt thereof. Both the R and the S enantiomers are provided.

As with the A group in formula II, in formula III the Z group can include additional L and Y groups. Compounds according to formula III also include those in which n=0 (no Y linkage) and L₃ is a single bond. In these compounds, the Z group is bonded directly to the dithiolane ring. Exemplary Z groups include imines and guanidines, as well as groups of the formula —NR3R4.

Compounds according to formula III in which W₁ and W₂ jointly denote—S—S—are typically non-metabolized forms. The present invention also contemplates and encompasses metabolites of such compounds, in particular, metabolites in which one or both sulfur atoms are oxidized. Such compounds include those compounds of formula III in which W₁ and W₂ jointly denote—S(O)—S—or —S—S(O)— (thiolsulfinates), or individually denote a group including an oxidized sulfur atom, such as an SO₃H group.

Other illustrative analogs of lipoic acid contemplated by the present invention include 5-(1,1-dithiolane-3-yl)pentanoic acid (alpha-lipoic acid); 5-(5-methyl-1,2-dithiolane-3-yl)pentanoic acid; ethyl-1,2-dithiolane-3-yl-formate; 4-ter.butyl-5(1,2-dithiolane-3-yl)pentanoamide; 2-(1,2-dithiolane-3-yl)acetic acid; 3-(1,2-dithiolane-3-yl)propionic acid; 6-(1,2-dithiolane-3-yl)hexanoic acid; 9-(1,2-dithiolane-3-yl)nonanoic acid; 4-methyl-4-ethyl-4-(1,2-dithiolane-3-yl)butyric acid; oxidized 5-(1,2-dithiolane-3-yl)pentanoic acid (beta-lipoic acid); 6,8-dimercaptooctanoic acid; 6,8-dimercaptononanoic acid; ethyl-2,4-dimercaptobutyrate; 4-tert.butyl-6,8-dimercaptooctanoamide; 3,5-dimercaptopentanoic acid; 4,6-dimercaptohexanoic acid; 7,9-dimercaptononanoic acid; 10,12-dimercaptododecanoic acid; 4-methyl-4-ethyl-5,7-dimercaptoheptanoic acid; and the sodium salt of 6,8-dimercaptooctanoic acid.

As described herein, the inclusion of lipoic acid in plant transformation media may advantageously be used with any plant species. Particularly preferred species for practice of the present invention include tomato, cotton, potato, wheat, corn, rice, and oilseeds, such as soybean, sunflower and oilseed rape species.

The present invention provides for obtaining a fertile transgenic plant and a method for the transformation of plant cells or tissues and regeneration of the transformed cells or tissues into a fertile, differentiated transformed plant. Although various transformation systems are well known to those skilled in the art, a brief description of the process is provided below.

Typically, to initiate a transformation process in accordance with the present invention, it is first necessary to select the genetic components desired to be inserted into the plant cells or tissues. Genetic components may include any nucleic acid that is introduced into a plant cell or tissue using the method according to the invention. Genetic components can include non-plant DNA, plant DNA, or synthetic DNA.

In a preferred embodiment, the genetic components are incorporated into a DNA composition such as a recombinant, double-stranded plasmid or vector molecule comprising at least one or more of the following types of genetic components: (a) a promoter that functions in plant cells to cause the production of an RNA sequence, (b) a structural DNA sequence that causes the production of an RNA sequence that encodes a desired protein or polypeptide, and (c) a 3′ non-translated DNA sequence that functions in plant cells to cause the addition of polyadenylated nucleotides to the 3′ end of the RNA sequence. The vector may also contain a number of genetic components to facilitate transformation of the plant cell or tissue and regulate expression of the desired gene(s).

The genetic components are typically oriented so as to express a mRNA, which in one embodiment can be translated into a protein. The expression of a plant structural coding sequence (a gene, cDNA, synthetic DNA, or other DNA) that exists in double-stranded form involves transcription of messenger RNA (mRNA) from one strand of the DNA by RNA polymerase enzyme and subsequent processing of the mRNA primary transcript inside the nucleus. This processing involves a 3′ non-translated region that adds polyadenylated nucleotides to the 3′ ends of the mRNA.

Means for preparing plasmids or vectors containing the desired genetic components are well known in the art. Vectors typically consist of a number of genetic components, including but not limited to regulatory elements such as promoters, leaders, introns, and terminator sequences. Regulatory elements are also referred to as cis- or trans-regulatory elements, depending on the proximity of the element to the sequences or gene(s) they control.

A region of DNA usually referred to as the “promoter” regulates transcription of DNA into mRNA. The promoter region contains a sequence of bases that signals RNA polymerase to associate with the DNA and to initiate the transcription into mRNA using one of the DNA strands as a template to make a corresponding complementary strand of RNA.

A number of promoters that are active in plant cells have been described in the literature. Such promoters would include, but are not limited to, the nopaline synthase (NOS) and octopine synthase (OCS) promoters, which are carried on tumor-inducing plasmids of Agrobacterium tumefaciens; the caulimovirus promoters such as the cauliflower mosaic virus (CaMV) 19S and 35S promoters and the figwort mosaic virus (FMV) 35S promoter; the enhanced CaMV35S promoter (e35S); and the light-inducible promoter from the small subunit of ribulose bisphosphate carboxylase (ssRUBISCO, a very abundant plant polypeptide). All of these promoters have been used to create various types of DNA constructs that have been expressed in plants. See, for example, PCT publication WO 84/02913. Promoter hybrids can also be constructed to enhance transcriptional activity or to combine desired transcriptional activity, inducibility, and tissue or developmental specificity.

Thus, promoters that function in plants may be inducible, viral, synthetic, constitutive as described, temporally regulated, spatially regulated, and/or spatio-temporally regulated. Other promoters that are tissue-enhanced, tissue-specific, or developmentally regulated are also known in the art and envisioned to have utility in the practice of this invention. Useful promoters may be obtained from a variety of sources such as plants and plant DNA viruses. As described below, it is preferred that the particular promoter selected should be capable of causing sufficient expression to result in the production of an effective amount of the gene product of interest.

The promoters used in the DNA constructs (i.e., chimeric/recombinant plant genes) of the present invention may be modified, if desired, to affect their control characteristics. Promoters can be derived by means of ligation with operator regions, random or controlled mutagenesis, etc. Furthermore, the promoters may be altered to contain multiple “enhancer sequences” to assist in elevating gene expression.

The mRNA produced by a DNA construct of the present invention may also contain a 5′ non-translated leader sequence. This sequence can be derived from the promoter selected to express the gene and can be specifically modified so as to increase translation of the mRNA. The 5′ non-translated regions can also be obtained from viral RNAs, from suitable eukaryotic genes, or from a synthetic gene sequence. Such “enhancer” sequences may be desirable to increase or alter the translational efficiency of the resultant mRNA. The present invention is not limited to constructs wherein the non-translated region is derived from both the 5′ non-translated sequence that accompanies the promoter sequence. Rather, the non-translated leader sequence can be derived from unrelated promoters or genes. Other genetic components that serve to enhance expression or affect transcription or translational of a gene are also envisioned as genetic components.

The 3′ non-translated region of the chimeric constructs should contain a transcriptional terminator, or an element having equivalent function, and a polyadenylation signal, which functions in plants to cause the addition of polyadenylated nucleotides to the 3′ end of the RNA. Examples of suitable 3′ regions are (1) the 3′ transcribed, non-translated regions containing the polyadenylation signal of Agrobacterium tumor-inducing (Ti) plasmid genes, such as the nopaline synthase (NOS) gene, and (2) plant genes such as the soybean storage protein genes and the small subunit of the ribulose-1,5-bisphosphate carboxylase (ssRUBISCO) gene. An example of a preferred 3′ region is that from the ssRUBISCO E9 gene from pea.

Typically, DNA sequences located a few hundred base pairs downstream of the polyadenylation site serve to terminate transcription. The DNA sequences are referred to herein as transcription-termination regions. The regions are required for efficient polyadenylation of transcribed messenger RNA (mRNA) and are known as 3′ non-translated regions. RNA polymerase transcribes a coding DNA sequence through a site where polyadenylation occurs.

In many transformation systems, it is necessary for the transformation vector to contain a selectable, screenable, or scorable marker gene. These genetic components are also referred to herein as functional genetic components, as they produce a product that serves a function in the identification of a transformed plant, or a product of desired utility. The DNA that serves as a selection device functions in a regenerable plant tissue to produce a compound that confers upon the plant tissue resistance to an otherwise toxic compound. Genes of interest for use as a selectable, screenable, or scorable marker would include, but are not limited to, β-glucuronidase (GUS), green fluorescent protein (GFP—), luciferase (LUX), antibiotics like kanamycin (Dekeyser et al., Plant Physiol., 90:217-223, 1989), and herbicides like glyphosate (Della-Cioppa et al., Bio/Technology, 5:579-584, 1987). Other selection devices can also be implemented including but not limited to tolerance to phosphinothricin, bialaphos, and positive selection mechanisms (Joersbo et al., Mol. Breed., 4:111-117, 1998) and would still fall within the scope of the present invention.

The present invention can be used with any suitable plant transformation plasmid or vector containing a selectable or screenable marker and associated regulatory elements as described, along with one or more nucleic acids (a structural gene of interest) expressed in a manner sufficient to confer a particular desirable trait. Examples of suitable structural genes of interest envisioned by the present invention would include, but are not limited to, genes for insect or pest tolerance, herbicide tolerance, genes for quality improvements such as yield, nutritional enhancements, environmental or stress tolerances, or any desirable changes in plant physiology, growth, development, morphology, or plant product(s).

Alternatively, the DNA coding sequences can effect these phenotypes by encoding a gene suppression construct. As used herein “gene suppression” means any of the well-known methods for suppressing a transcript or a protein from a gene including post transcriptional gene suppression and transcriptional suppression. Post transcriptional gene suppression is mediated by transcribed RNA having homology to a gene targeted for suppression. The RNA transcribed from the suppressing transgene can be in the sense orientation to effect what is called co-suppression, in the anti-sense orientation to effect what is called anti-sense suppression or in both orientations producing a double-stranded RNA to effect what is called RNA interference (RNAi). Transcriptional suppression is mediated by a transcribed double-stranded RNA having homology to promoter DNA sequence to effect what is called promoter trans suppression.

More particularly, post transcriptional gene suppression by anti-sense oriented RNA to regulate gene expression in plant cells is disclosed in U.S. Pat. No. 5,107,065 (Shewmaker et al.) and U.S. Pat. No. 5,759,829 (Shewmaker et al.). Post transcriptional gene suppression by sense-oriented RNA to regulate gene expression in plants is disclosed in U.S. Pat. No. 5,283,184 (Jorgensen et al.) and U.S. Pat. No. 5,231,020 (Jorgensen et al.). Post transcriptional gene suppression by double-stranded RNA to suppress genes in plants by RNAi is disclosed in PCT Publication WO 99/53050 (Waterhouse et al.) using recombinant DNA constructs comprising sense-oriented and anti-sense-oriented elements of a targeted gene in separate transcription units or in a single transcription unit. See, also PCT Publication WO 99/49029 (Graham et al.), U.S. Publication 2003/0175965 A1 (Lowe et al.), U.S. patent application Ser. No. 10/465,800 (Fillatti), and U.S. Pat. No. 6,506,559 (Fire et al.). See, also Titia Sijen et al., The Plant Cell, 8:2277-2294, December 1996, which discloses the use of constructs carrying inverted repeats of a cowpea mosaic virus gene in transgenic plants to mediate virus resistance. Another DNA construct for RNAi gene suppression comprising a singly-oriented gene element bordered by oppositely-oriented promoters is disclosed in U.S. Publication 2003/0061626 A1 (Plaetinck et al.) and U.S. Pat. No. 6,326,193. See, also U.S. patent application Ser. No. 10/393,347, which discloses constructs and methods for simultaneously expressing one or more recombinant genes while simultaneously suppressing one or more native genes in a transgenic plant. See also U.S. Pat. No. 6,448,473, which discloses multigene expression vectors for use in plants. All of the above-described patents, applications and international publications disclosing materials and methods for post transcriptional gene suppression in plants are incorporated herein by reference.

A preferred method of post transcriptional gene suppression in plants employs either sense-oriented or anti-sense-oriented, transcribed RNA that is stabilized, e.g., with a terminal hairpin structure. A preferred DNA construct for effecting post transcriptional gene suppression is transcribed to a segment of anti-sense oriented RNA having homology to a gene targeted for suppression, where the anti-sense RNA segment is followed at the 3′ end by a contiguous, complementary, shorter segment of RNA in the sense orientation. The use of self-stabilized anti-sense RNA oligonucleotides in plants is disclosed in PCT Publication WO 94/01550 (Agrawal et al.). See also PCT Publication WO 98/05770 (Werner et al.), where the anti-sense RNA is stabilized by hairpin forming repeats of poly(CG) nucleotides. See also U.S. Publication 2002/0048814 A1 (Oeller), where sense or anti-sense RNA is stabilized by a poly(T)-poly(A) tail. See also U.S. Publication 2003/0018993 A1 (Gutterson et al.), where sense or anti-sense RNA is stabilized by an inverted repeat of a subsequence of an NOS gene. See also U.S. Publication 2003/0036197 A1 (Glassman et al.), where RNA having homology to a target is stabilized by two complementary RNA regions. All of the above-described patents, applications and international publications disclosing materials and methods for employing stabilized RNA and its use in gene suppression in plants are incorporated herein by reference.

Transcriptional suppression such as promoter trans suppression can be effected by a expressing a DNA construct comprising a promoter operably linked to inverted repeats of promoter DNA for a target gene. Constructs useful for such gene suppression mediated by promoter trans suppression are disclosed by Mette et al., The EMBO Journal, 18(1):241-148, 1999, and by Mette et al., The EMBO Journal, 19(19):5194-5201, 2000, both of which are incorporated herein by reference.

Exemplary nucleic acids that may be introduced by the methods encompassed by the present invention include, for example, DNA sequences or genes from another species, or even genes or sequences that originate with or are present in the same species but are incorporated into recipient cells by genetic engineering methods rather than classical reproduction or breeding techniques. The term exogenous, however, is also intended to refer to genes that are not normally present in the cell being transformed or to genes that are not present in the form, structure, etc., as found in the transforming DNA segment or to genes that are normally present but a different expression is desirable. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

In light of this disclosure, numerous other possible selectable or screenable marker genes, regulatory elements, and other sequences of interest will be apparent to those of skill in the art. Therefore, the foregoing discussion is intended to be exemplary rather than exhaustive.

Several technologies for the introduction of DNA into cells are well known to those of skill in the art and can be divided into categories including but not limited to: (1) chemical methods; (2) physical methods such as microinjection, electroporation and particle bombardment; (3) viral vectors; (4) receptor-mediated mechanisms; and (5) Agrobacterium-mediated plant transformation methods.

After the construction of the plant transformation vector or construct, the nucleic acid molecule, prepared as a DNA composition in vitro, is introduced into a suitable host such as E. coli and mated into another suitable host such as Agrobacterium, or directly transformed into competent Agrobacteria. These techniques are well-known to those of skill in the art and have been described for a number of plant systems including soybean, cotton, and wheat.

Those of skill in the art would recognize the utility of Agrobacterium-mediated transformation methods. Preferred strains would include, but are not limited to, Agrobacterium tumefaciens strain C58, a nopaline strain that is used to mediate the transfer of DNA into a plant cell; octopine strains, such as LBA4404; or agropine strains, e.g., EHA101, EHA105, or EHA109. The use of these strains for plant transformation has been reported, and the methods are familiar to those of skill in the art.

The present invention can be used with any regenerable cell or tissue. Those of skill in the art recognize that regenerable plant tissue generally refers to tissue that after insertion of exogenous DNA and appropriate culture conditions can form into a differentiated plant. Such tissue can include, but is not limited to, callus tissue, hypocotyl tissue, cotyledons, meristematic tissue, roots, and leaves. For example, regenerable tissues can include calli or embryoids from anthers, microspores, inflorescences, and leaf tissues. Other tissues are also envisioned to have utility in the practice of the present invention, and the desirability of a particular explant for a particular plant species is either known in the art or may be determined by routine screening and testing experiments whereby various explants are used in the transformation process and those that are more successful in producing transgenic plants are identified.

In one embodiment of the present invention, 7-day-old tomato cotyledons are used as the starting explant material. In another embodiment, pieces of potato stems are used as the starting explant material. In yet another embodiment, cotton callus is used as the starting explant material. Immature embryos of wheat are used in a further embodiment.

Once the regenerable plant tissue is isolated, the next step of the method is introducing the genetic components into the plant tissue. This process is also referred to herein as “transformation.” The plant cells are transformed and each independently transformed plant cell is selected. The independent transformants are referred to as plant cell lines or “events”.

Methods for transforming dicots, primarily by use of Agrobacterium tumefaciens, and obtaining transgenic plants have been published for a number of crops including cotton, soybean, Brassica, and peanut.

Successful transformation of monocotyledonous plants describing the use of electroporation, particle bombardment, and/or Agrobacterium based methods have also been reported. Transformation and plant regeneration have been achieved and reported at least in asparagus, barley, maize, oat, rice, sugarcane, tall fescue, and wheat.

The present invention finds particular use in Agrobacterium-mediated transformation processes. Agrobacterium-inoculated explants are typically cultured on an appropriate co-culture medium to allow for transfer of the genetic component containing the gene-of-interest to be introduced into the plant cells/tissue for incorporation into its genome. Appropriate co-culture media is typically known for each culture system or can be determined by one of skill in the art. In accordance with the present invention, the co-culture media may contain an effective amount of lipoic acid or an analog thereof.

The Agrobacterium-inoculated explants are then typically cultured on an appropriate medium containing an agent to inhibit Agrobacterium growth. This media is usually refereed to as a delay media or a selection media, as described below. The Agrobacterium-inoculated explants are cultured on such a media generally from one to fourteen days, preferably from two to seven days. Those of skill in the art are aware of the appropriate media components to inhibit Agrobacterium growth. Such media components would include, but are not limited to, antibiotics such as carbenicillin or cefotaxime.

After the culture step to inhibit Agrobacterium growth, and preferably before the explants can be placed on selective media, they can be analyzed for efficiency of DNA delivery by a transient assay that detects the presence of a gene contained on the transformation vector, including, but not limited to, a screenable marker gene such as the gene that codes for β-glucuronidase (GUS). The total number of blue spots (indicating GUS expression) for a selected number of explants is used as a positive correlation of DNA transfer efficiency.

In a preferred embodiment, after incubation on non-selective media containing the antibiotics to inhibit Agrobacterium growth without selective agents (delay medium), the explants are cultured on selective growth media including, but not limited to, a callus-inducing media containing a selective agent. Typical selective agents have been described and include, but are not limited to, antibiotics such as geneticin (G418), paromomycin, kanamycin, or other chemicals such as glyphosate. Delay media or selection media may also contain an effective amount of lipoic acid or an analog thereof. The plant tissue cultures surviving the selection media are subsequently transferred to a regeneration media suitable for the production of transformed plantlets. Regeneration can be carried out over several steps. Regeneration media at any step may contain an effective amount of lipoic acid or an analog thereof. Those of skill in the art are aware of the numerous types of media and transfer requirements that can be implemented and optimized for each plant system for plant transformation and regeneration. Consequently, such media and culture conditions disclosed in the present invention can be modified or substituted with nutritionally equivalent components, or similar processes for selection and regeneration, and still fall within the scope of the present invention.

The transformants produced are subsequently analyzed to determine the presence or absence of a particular nucleic acid of interest contained on the transformation vector. Molecular analyses can include, but are not limited to, Southern blots (Southern, Mol. Biol., 98:503-517, 1975) or PCR (polymerase chain reaction) analyses. These and other well known methods can be performed to confirm the stability of the transformed plants produced by the methods disclosed. These methods are well known to those of skill in the art and have been reported (see, for example, Sambrook et al., Molecular Cloning: A Laboratory Manual, Second Edition, Cold Spring Harbor Laboratory Press, Cold Spring Harbor, N.Y., 1989).

The previous discussion is merely a broad outline of standard transformation and regeneration protocols. One of skill in the art knows that specific crops and specific protocols can vary somewhat from the broad outline. A variety of medias can be used in each system as well. Those of skill in the art are familiar with the variety of tissue culture media that, when supplemented appropriately, support plant tissue growth and development. These tissue culture media can either be purchased as a commercial preparation or custom prepared and modified by those of skill in the art. Examples of such media would include, but are not limited to, Murashige and Skoog (Murashige and Skoog, Physiol. Plant, 15:473-497, 1962), N6 (Chu et al., Scientia Sinica 18:659, 1975), Linsmaier and Skoog (Linsmaier and Skoog, Physio. Plant., 18: 100, 1965), Uchimiya and Murashige (Uchimiya and Murashige, Plant Physiol. 15:473, 1962), Gamborg's media (Gamborg et al., Exp. Cell Res., 50:151, 1968), D medium (Duncan et al., Planta, 165:322-332, 1985), McCown's Woody plant media (McCown and Lloyd, HortScience 16:453, 1981), Nitsch and Nitsch (Nitsch and Nitsch, Science 163:85-87, 1969), and Schenk and Hildebrandt (Schenk and Hildebrandt, Can. J. Bot. 50:199-204, 1972) or derivations of these media supplemented accordingly. Those of skill in the art are aware that media and media supplements such as nutrients and growth regulators for use in transformation and regeneration are usually optimized for the particular target crop or variety of interest.

### EXAMPLES

The following examples further illustrate the present invention. They are in no way to be construed as a limitation in scope and meaning of the claims.

### Example 1

### MicroTomato Transformation

This example describes the use of lipoic acid in an Agrobacterium-based tomato transformation process. MicroTom (a cultivar of tomato species: Lycopersicon esculentum) seeds from Tomato Growers (Ball Seed Co., West Chicago, Ill. USA) were surface-sterilized by first soaking for 10 min in sterile deionized water, removing the water, and then adding enough 70% ethanol to immerse the seeds for 1 min while stirring vigorously and then aspirating the ethanol. The seeds were rinsed 2× with sterile deionized water and then soaked in 25% Chlorox with 1-2 drops of Tween 20 for 10 min while stirring frequently. The seeds were rinsed 3× with sterile deionized water. The seeds were germinated in phytatrays containing Davis Germination medium (4.3 g/L MS salts, 2 ml/L Gamborg's B-5 500× salts, 20 g/L sucrose and 10 g/L agar) (approximately 120 seeds/container), and the phytatrays were wrapped with aluminum foil. The phytatray cultures were maintained at 28° C. under the dark for 5 days. After 5 days, the phytatray cultures were unwrapped and grown at 24° C. under a 16 h photoperiod for about 44 h.

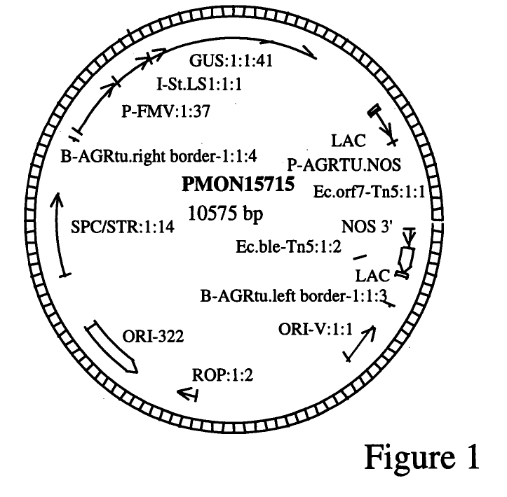

Agrobacterium containing the desired transformation vector with a selectable marker (nptII for kanamycin selection as shown in FIG. 1) was streaked on a fresh LBssck plate (LB, 200 mL; spectinomycin (50 mg/mL stock), 200 μL; streptomycin (50 mg/mL stock), 200 μL; kanamycin (50 mg/mL stock), 200 μL; chloramphenicol (CAP, 25 mg/mL stock), 200 μL) from a frozen glycerol stock, and grown at about 28° C. for 3-4 days. One day before inoculation, a loopful of the bacterium cells from the freshly streaked plate was inoculated into 2 mL of LB+(LB, 200 mL; spectinomycin or streptomycin (50 mg/mL stock), 400 μL; kanamycin (50 mg/mL stock), 200 μL; chloramphenicol (CAP, 25 mg/mL stock), 200 μL) without acetosyringone, and grown at about 28° C. on a rotator at the speed between medium to high overnight. Then, 0.2 mL of the overnight bacterium culture was inoculated into 2 mL of LB+0.2 mM acetosyringone, and grown at about 28° C. on a rotator at the speed between medium to high for 4 hours.

Four hours after the sub-culture, the bacterium growth phase was measured using the spectrophotometer. The final O.D.₆₆₀ of the bacterium culture was between about 0.9 and 1.4. The bacterium culture was diluted by adding the bacterium culture to TXD medium in a sterile tube based on the volume to get the working O.D.₆₆₀=0.1.

Cotyledon explants were isolated from seedlings 7 days after sowing seeds on Davis Germination medium. Cotyledon explants were prepared by bathing them in 6 mL of TXD liquid medium (4.3 g/L MS salts, 2 mL/L Gamborg's B-5 500× salts, 4 mg/L pCPA (p-chlorophenoxyactic acid), 0.005 mg/L kinetin, and 30 g/L sucrose) to prevent desiccation and trimming them at both ends using a #15 feather blade. Immediately after the trimming, the cotyledon explants were gently placed on “feeder plates” containing UC2PC medium (2.2 g/L MS salts, 2.2 mL/L, Gamborg's B-5 500× salts, 0.2 mg/L 2,4-D, 4 mg/L pCPA, 0.1 mg/L kinetin, 30 g/L sucrose, 0.2 g/L potassium phosphate, 10 g/L agar, 8 mL/L acetosyringone (4.91 mg/mL), and 4 mL galacturonic acid (53.05 mg/mL)). The “feeder plates” are made by overlaying 2 mL of tobacco suspension cells and a sterile Whatman filter on the plates containing UC2PC medium. The cotyledon explants were gently poked with a sharp forceps (ASIM5311, Stainless French, made in Switzerland). Usually six to ten pokes were made per explant depending the size of cotyledons. Right after poking, the cotyledons were inoculated with Agrobacterium containing pMON15715 (FIG. 1) by adding 3 mL of the adjusted Agrobacterium solution on top of the cotyledons. The plates were incubated for 10 min at room temperature, and the Agrobacterium solution was aspirated with a sterile pipette. The cotyledons were then co-cultured for 2 days at 23-24° C. under a 16-h photoperiod. The co-culture plates were placed in plastic bags to keep the humidity, which is preferable for MicroTom growth.

For Kanamycin Selection

Regeneration

Induction and Elongation Stage

Two days after co-cultivation, cotyledons were transferred to shoot induction medium SI-2 (4.3 g/L MS salts, 2 mL/L, Gamborg's B-5 500× salts, 30 g/L sucrose, 10 g/L agar, 500 mg/L carbenicillin, 100 mg/L cefotaxime, 0.1 mg/L IAA, and 2 mg/L zeatin riboside) supplemented with 10 μM of lipoic acid and 100 mg/L kanamycin. Three to four weeks after the induction stage, the cotyledons formed buds or shoots. Cotyledons with buds or shoots were separated by individual event and transferred to, shoot elongation medium E2 (SI-2 medium without IAA), supplemented with 100 mg/L kanamycin for two to four weeks. All culture plates were placed in a culture box, placed inside a plastic bag, and cultured at 23-24° C. under a 16 h photoperiod.

Rooting Stage and Rooting Screening Method

When shoots elongated (about 2 to 3 cm in length), they were excised from cotyledons and transferred to rooting medium MTR2 (2.2 g/L MS salts, 2.0 mL/L Gamborg's B-5 500× salts, 30 g/L sucrose, 10 g/L agar, 2 mL/L carbenicillin (250 mg/mL), 0.75 mL/L cefotaxime (100 mg/mL), and 1 mL/L IBA (1 mg/mL)) with 40 mg/L kanamycin at a density of one to two shoots per plate for about three to four weeks. Culture plates were placed in a plastic bag and cultured at 23-24° C. under a 16-h photoperiod.

For Glyphosate Selection

Regeneration

First Transfer: Delay

Two days after co-cultivation, cotyledons were transferred to delay medium D1 or D2 (both contain 4.3 g/L MS salts, 2 mL/L, Gamborg's B-5 500× salts, 30 g/L sucrose, 10 g/L agar, 500 mg/L carbenicillin, 100 mg/L cefotaxime and 2 mg/L zeatin riboside; D2 in addition contains 0.5 mg/L IAA) containing 10 μM lipoic acid for 7 days. All culture plates were placed in a culture box, placed inside a plastic bag, and cultured at 23-24° C. under a 16-h photoperiod.

Second Transfer: Selection/Shoot Induction

Cotyledon explants were transferred to shoot induction medium SI-2 supplemented with 0.03 mM glyphosate and 10 μM lipoic acid. All culture plates were placed in a culture box placed inside a plastic bag and cultured at 23-24° C., under a 16-h photoperiod

Third Transfer: Selection/Elongation

Three to four weeks after induction stage, the cotyledons formed buds or shoots. Cotyledons with buds or shoots were separated by individual event and transferred to shoot elongation medium CPE1 (SI-2 media minus IAA and with 0.5 mg/L zeatin riboside), supplemented with 0.03 mM glyphosate for two to four weeks. All culture plates were placed in a culture box, placed inside a plastic bag, and cultured at 23-24° C. under a 16-h photoperiod.

Fourth Transfer: Rooting

When shoots elongated (about 2 to 3 cm in length), they were excised from cotyledons and transferred to rooting medium CPR (Gibco MS, 2.2 g/L; Gamborg's B-5 500×, 2 mL/L; sucrose, 30 g/L; gum agar, 10 g/L; carbenicillin, 500 mg/L; cefotaxime, 75 mg/L; IBA, 1 mg/L; pH=5.8), at a density of one to two shoots per plate for about three to four weeks. All culture plates were placed in a plastic bag and cultured at 23-24° C. under a 16 h photoperiod.

Evaluation of plants after transformation with pMON15715 (FIG. 1) as described above was carried out in multiple ways. About five days after selection, tissue browning severity within and around the poked region on cotyledon explants was measured under a dissecting microscope. Low tissue browning severity was determined as less than 30% of the pokes made on each cotyledon explant that produced browning tissue and the high tissue browning severity was determined as more than 30% of the pokes made on each explant that generated browning tissue 5 days after culturing on selection medium. All the treatments of lipoic acid at concentrations of 5, 10, 50 and 100 μM had 1.5- to 1.8-fold increases of the percentage of explants scored as low tissue browning severity, respectively, compared to the treatment without lipoic acid (Table 1). The treatment without lipoic acid had a higher percentage of explants that scored as high tissue browning severity than those of the lipoic acid treatments (63.9% in the treatment without lipoic acid vs. 33.9 to 46% in the treatments with lipoic acid). The results indicated that using lipoic acid had about twofold reduction of tissue browning at all the concentrations tested.

TABLE 1
Effect of lipoic acid on tissue browning
after 5 days on selection media.

| Lipoic Acid | % low | % high |
|---|---|---|
| Conc. (μM) | browning | browning |
| 0 | 36.1 | 63.9 |
| 5 | 65.1 | 34.9 |
| 10 | 66.1 | 33.9 |
| 50 | 65.6 | 34.4 |
| 100 | 54 | 46 |

At the same time the tissue browning severity was measured, transient expression of the gus gene was scored. High transient expression was defined as an explant having more than 30% of the pokes that produced GUS blue spots. Low transient expression was defined as an explant having less than 30% of the pokes that produced GUS blue spots about 5 days after culturing on selection medium. Nontransient expression was determined as an explant having no GUS blue spot. With the lipoic acid treatments at the concentrations of 10 and 50 μM, the percentage of explants having high transient expression doubled from 30% (non-lipoic acid treatment) up to 60.9% (Table 2). The treatments with lipoic acid at concentrations of 5, 10 and 50 μM produced the higher percentages of 42.9%, 60.9% and 56.5%, respectively, of explants having high transient expression than those of non-lipoic acid treatment (30%) and lipoic acid treatment at the concentration of 100 μM (34.8%). When lipoic acid was used in the selection process at the concentrations of 10, 50 and 100 μM, 100% of explants transiently expressed the GUS gene, but without lipoic acid or lipoic acid at the concentration of 5 μM, 20% and 9.5% of explants had no GUS transient expression.

TABLE 2
Effect of lipoic acid on transient GUS expression
after 5 days on selection media.

| Lipoic Acid conc. | % High transient | % Low transient | % No transient |
|---|---|---|---|
| (μM) | expression | expression | expression |
| 0 | 30 | 50 | 20 |
| 5 | 42.9 | 47.6 | 9.5 |
| 10 | 60.9 | 39.1 | 0 |
| 50 | 56.5 | 43.5 | 0 |
| 100 | 34.8 | 65.2 | 0 |

The percentage of transgenic plants produced per explant had a 2.7- to 4.3-fold increase when lipoic acid was used at all concentrations tested (Table 3). The lipoic acid at a concentration of 10 μM generated the highest percentage (178.7% versus 40.1% with non-lipoic acid treatment) of transgenic plants produced per explant. The lipoic acid at concentrations of 5, 50 and 100 μM produced higher percentages of 155.1%, 144.1% and 112.1%, respectively, of transgenic plants produced per explant than that of 40.1% of non-lipoic acid treatment. The percentage of transgenic plants produced per explant increased with the reduction of tissue browning and the increase of high transient expression by using lipoic acid.

TABLE 3
Effect of lipoic acid on cell browning (5 days after selection),
transient GUS expression (5 days after selection), transformation
efficiency (TE) (percentage of transgenic events produced
per explant), and stable transformation efficiency
(percentage of transgenic plants produced per explant).

| Lipoic Acid | % low | % high transient | | |
|---|---|---|---|---|
| conc. (μM) | browning | expression | TE | Efficiency |
| 0 | 36.1 | 30 | 27.9 | 41.4 |
| 5 | 65.1 | 42.9 | 78.3 | 155.1 |
| 10 | 66.1 | 60.9 | 94.1 | 178.7 |
| 50 | 65.6 | 56.5 | 76.4 | 144.1 |
| 100 | 54 | 34.8 | 49.7 | 112.1 |

The percentage of independent transgenic plants (transgenic events) produced per explant had a 1.8- to 3.4-fold increase by using lipoic acid at all concentrations tested compared with non-lipoic acid treatment (Table 3). Lipoic acid at 10 μM produced the highest percentage (94.1%) of transgenic events produced per explant among non-lipoic acid treatment and other lipoic acid treatments. The lipoic acid at concentrations of 5, 50 and 100 μM produced higher percentages of 78.3%, 76.4 and 49.7%, respectively, of transgenic events produced per explant than that of 27.9% of non-lipoic acid treatment. The results showed that lipoic acid increased both the percentage of transgenic plants produced per explant and the percentage of transgenic events produced per explant at all the concentrations tested. The best concentration, which resulted in both the highest percentage of transgenic plants produced per explant and percentage of transgenic events produced per explant, was 10 μM. When the lipoic acid concentration increased to 100 μM, both the percentage of transgenic plants produced per explant and the percentage of transgenic events produced per explant decreased compared with those with lipoic acid concentrations at 5, 10 and 50 μM.

As in most kanamycin-selection crop transformation systems, about 80 to 90% of shoots that are produced on selection media were non-transgenic shoots, termed escapes. To determine the effect of lipoic acid on escape production at the shoot stage, the number of shoots that did not root (escapes) and of shoots that rooted (transgenic shoots) on selection medium were measured. When lipoic acid was used at the concentrations of 5, 10, 50 and 100 μM, 54.6, 58.3, 55.4 and 57.1%, respectively, of shoots were escapes, but 88.1% of shoots were escapes when not using lipoic acid. All lipoic acid concentrations of 5, 10, 50 and 100 μM tested had a 1.5- to 1.6-fold reduction in escapes in comparison to non-lipoic acid treatment.

To determine the effect of lipoic acid on transgenic and non-transgenic shoot induction under selection pressure, shoot growth and development were measured. Lipoic acid at the concentrations of 0, 5, 10, 50 and 100 μM had 70.8, 68.9, 67.4, 61.2 and 25.6%, respectively, of explants producing shoots on selection medium (Table 4). The percentages of the explants producing shoots were not significantly different between lipoic acid treatments of 5, 10 and 50 μM and non-lipoic acid treatment, but the percentage significantly decreased from 70.8% (0 μM lipoic acid) to 25.6% when using lipoic acid at the concentration of 100 μM. Also, the number of total shoots (non-transgenic and transgenic shoots) generated per initial explant plated was 3.52 on 0 μM lipoic acid, and 3.53, 4.17, and 3.00, respectively, on 5, 10 and 50 μM lipoic acid, and 2.21 on 100 μM lipoic acid (Table 4). The results indicated that lipoic acid had no inhibition effect on the total transgenic and non-transgenic shoot induction in terms of the percentage of the initial explant responding and number of shoots produced per the initial explant plated when lipoic acid was used at concentrations from 5 μM to 50 μM. In addition, lipoic acid had a 1.2- to 1.7-fold increase in the number of shoots produced per the responding explant at the concentrations of 10 and 100 μM.

TABLE 4
Effect of lipoic acid on shoot regeneration and transgenic plant production.

| | | % of explants | | |
|---|---|---|---|---|
| | | producing shoots | | No. of total shoots |
| | No. of | (>1 cm | No. of total shoots | produced/resp. |
| Treatment | explants | in length) | produced/explant | explant |
| 0 μM lipoic acid | 48 | 70.8 | 3.52 | 4.97 |
| 5 μM lipoic acid | 45 | 68.9 | 3.53 | 4.93 |
| 10 μM lipoic acid | 46 | 67.4 | 4.17 | 6.19 |
| 50 μM lipoic acid | 49 | 61.2 | 3.00 | 4.83 |
| 100 μM lipoic acid | 43 | 25.6 | 2.21 | 8.64 |

There was no significant difference in the total number of non-transgenic and transgenic shoots produced on 0, 5, 10 and 50 μM lipoic acid media in terms of the percentage of the responding explant and number of the total shoots produced per explant plated. However, only 32.4% of responding explants produced transgenic plants when lipoic acid was not used. When lipoic acid was used at the concentrations of 5, 10, 50 and 100 μM, 78.6, 74.2, 67.9 and 93.8% of responding explants produced transgenic plants (Table 5). The percentages of the responding explants that generated transgenic plants increased from 2- to 4-fold with all lipoic acid treatments compared with the non-lipoic acid treatment (Table 5). Furthermore, among the total shoots produced on selection medium with lipoic acid at 0, 5, 10, 50 and 100 μM, 11.7, 43.9, 42.9, 48.0 and 50.6%, respectively, of them produced transgenic plants (Table 5). The percentage of transgenic plants produced per shoot had a 3.6- to 4.3-fold increase.

TABLE 5
Effect of lipoic acid on total shoot regeneration, transformation
efficiency and escape production.

| | | | % of resp. | | |
|---|---|---|---|---|---|
| | | % of explants | explant | No. of total | |
| | | producing | producing | shoots | |
| | No. of | shoots (>1 cm | transgenic | produced/ | % of transgenic |
| Treatment | explants | in length) | plants | explant | plants/shoot |
| 0 μM lipoic acid | 48 | 70.8 | 32.4 | 3.52 | 11.7 |
| 5 μM lipoic acid | 45 | 68.9 | 78.6 | 3.53 | 43.9 |
| 10 μM lipoic acid | 46 | 67.4 | 74.2 | 4.17 | 42.9 |
| 50 μM lipoic acid | 49 | 61.2 | 67.9 | 3.00 | 48.0 |
| 100 μM lipoic acid | 43 | 25.6 | 93.8 | 2.21 | 50.6 |

Lipoic acid increased the percentage of responding explants producing transgenic plants from 2- to 4-fold and the number of transgenic plants produced per responding explant from 4.0- to 7.4-fold, but not through increasing the total number of non-transgenic and transgenic shoots. The percentages of transgenic events per explant plated and transgenic plants per explant plated had 2.7- to 4.7-fold increases compared to those of non-lipoic acid treatment (Table 6). However, lipoic acid at the concentration of 100 μM significantly reduced the percentage of explants that developed shoots on selection medium from 70.8% (non-lipoic acid treatment) to 25.6%, although lipoic acid at the concentration of 100 μM generated the highest percentages of responding explants producing transgenic plants and the number of transgenic plants produced per the responding explants.

TABLE 6
Effect of lipoic acid on total shoot regeneration, transformation
efficiency and escape production.

| | | % of resp. | | | |
|---|---|---|---|---|---|
| | | explants | | | % of |
| | | producing | No. of transgenic | % of | transgenic |
| | No. of | transgenic | plants produced/ | transgenic | plants/ |
| Treatment | explants | plants | resp. explant | events/explant | explant |
| 0 μM lipoic acid | 48 | 32.4 | 0.59 | 27.9 | 41.1 |
| 5 μM lipoic acid | 45 | 78.6 | 2.25 | 78.3 | 155.1 |
| 10 μM lipoic acid | 46 | 74.2 | 2.65 | 94.1 | 178.7 |
| 50 μM lipoic acid | 49 | 67.9 | 2.35 | 76.4 | 144.1 |
| 100 μM lipoic acid | 43 | 93.8 | 4.38 | 49.7 | 112.1 |

In summary, it has been demonstrated that using lipoic acid in MicroTom transformation results in a 2-fold reduction of Agrobacterium infected plant tissue browning severity and tissue death, a 2-fold increase of the percentage of the explants having high level of transient expression, and subsequently a 6.5-fold increase of the stable transformation efficiency from 40% to up to 260%.

### Example 2

### Potato Transformation

This Example describes the use of lipoic acid in an Agrobacterium-based potato transformation protocol. Five days prior to inoculation, Agrobacterium containing the transformation vector of interest were streaked on a fresh LBssck plate (see media descriptions in Example 1) from the frozen glycerol stock, grown at about 28° C. for 3-4 days and stored in the refrigerator. About 20-22 hours before inoculation, 50 mL of liquid LBssck media (see media descriptions in Example 1) were dispensed into a 250-mL sterile flask. Using a sterile bacteriological loop, one loopful of bacteria from the LBssck plate was inoculated and agitated in the liquid LBssck. The 250-mL flasks were shaken at 25° C. overnight at medium to high speed. On inoculation day, 45 mL of MSO (4.4 g/L MS salts, 2 mL/L, Gamborg's B5 salts, 30 g/L sucrose) were dispensed into a 250 mL sterile flask. Using a sterile 5 mL pipette, 5 mL of the suspension was removed and dispensed into the flask containing the 45 mL of MSO and then agitated. The desired O.D. used for Agrobacterium transformation in potato was O.D.₆₆₀=0.1.

Potato Inoculation Protocol (See Media Composition Section for Media Ingredients)

The stock plant material to be used was collected from the growth chamber (16-hour light, 20° C.). The meristem was excised from the stock plants, and the cut end was placed in fresh PM (see media descriptions below) media, with ten meristems per plantcon. The leaves were removed from the potato stems by either cutting with a scalpel or pinching them off with forceps. After removing all the leaves in the plantcon, the stems were excised near the base and placed in an empty, sterile plantcon containing MSO. During this process, the plantcon(s) containing stems were kept closed except when adding more stems.

On the day of the inoculation, the stems were cut into 7 mm sections using a sterile scalpel and forceps. A 1/10 Agrobacterium solution was prepared by pipetting 4 mL of the Agrobacterium culture into 36 mL of MSO. The 1/10 agro solution was poured into the respective plates and swirled gently. A small amount of MSO was dispensed into the regeneration tissue and swirled. It was incubated for 15 min.

After 15 minutes, the Agrobacterium solution was removed with a vacuum aspirator. Using a sterile scoopula, the explants were scooped and spread onto the coculture plates with a piece of sterile 8.5 cm Whatman filter paper placed in the middle, about 100 explants per plate. The plates were placed in a bag and stored in a warm room (16/8 hour light, 19° C.) for two days.

After the two-day co-culture period, explants were placed on the appropriate selection media corresponding to the selectable marker provided in the transformation vector.

For Kanamycin Selection:

The explants were placed on the Z1 kanamycin media at 20 explants per plate. The plates were placed in a bag and stored in a warm room (16-hour light, 19° C.) for one week.

For Glyphosate Selection:

The explants were placed on the Z1 nonselection media at 20 explants per plate. The plates were placed in a bag and stored in a warm room (16-hour light, 19° C.). The glyphosate constructs were transferred to selection medium in 3 days.

In three days, the explants were transferred to Z1 glyphosate selection media at 20 explants per plate. The plates are bagged and placed in the warm room for one week.

After one week, the explants that were on Z1 glyphosate media were transferred to Z2 glyphosate media at 20 explants per plate. The plates were bagged and placed back in the warm room for 4 weeks. They remained on-this selection media for four weeks.

After two weeks, the experiment was checked for any shoots at this point. Shoots that were originating directly from the callusing end of the explant were excised. The shoots were placed on rooting media containing either kanamycin or glyphosate depending upon the selection used in the experiment and placed in the growth chamber. These were checked for rooting in two weeks. Any that were rooting were cut into tubes. This process was repeated after 2 more weeks. Any non-rooting plants were discarded after 4 weeks.

Lipoic Acid Study

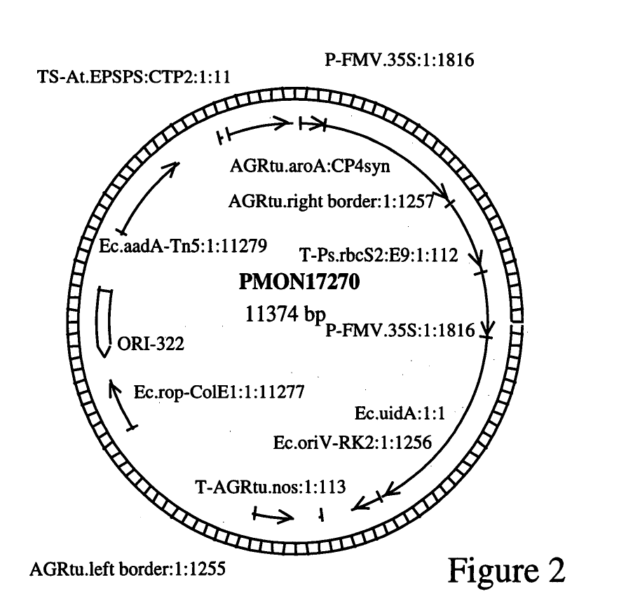

The lipoic acid study was conducted using the glyphosate transformation protocol as outlined above. The cultivar used for the procedure was Ranger Russet, which was grown under sterile conditions for three weeks. The solid media used in the experiment was prepared and had the appropriate test levels of lipoic acid added to perform the experiment. (Please refer to the media list for compositions.) The Agrobacterium vector used for this experiment was pMON17270 (FIG. 2), which is a CP4/GUS vector (conferring glyphosate resistance and a visual marker). The purpose for using a GUS vector was to have a quick method of screening all shoots and a random selection of callus to determine if the GUS reporter gene was present. Two thousand explants were inoculated and divided into five treatments for a total of 400 explants per treatment. The treatments corresponded to the lipoic acid level in the media (please refer to the media list for compositions). The experiment was conducted using the standard glyphosate protocol with all transfers occurring at the appropriate timepoints. The results obtained are outlined in Table 7. Previous experiments had shown that the optimal lipoic acid concentration was between 5 and 10 μM.

TABLE 7
Effect of lipoic acid on transformation in
potato (TE = transformation efficiency).

| lipoic acid | | | | | |
|---|---|---|---|---|---|
| μM | #explants | shoots | #rooted | % TE | % escape |
| 6 | 400 | 27 | 18 | 4.5 | 33.0 |
| 7 | 400 | 25 | 16 | 4.0 | 36.0 |
| 8 | 400 | 89 | 75 | 19.0 | 16.0 |
| 9 | 400 | 89 | 70 | 18.0 | 21.0 |
| control | 400 | 20 | 10 | 3.0 | 50.0 |

The shoots from each treatment were considered transgenic if they met BOTH of the following criteria:

1. They successfully rooted on rooting media containing the standard glyphosate levels used in the experiment.

2. They passed the GUS screen.

The typical transformation efficiency for potato transformation with glyphosate selection can range from 5-10% depending upon the gene of interest, and the control in this experiment as shown in Table 7 was 3%. Lipoic acid at 6 and 7 μM showed little effect on transformation efficiency and escapes. Lipoic acid at 8 μM and 9 μM showed a sixfold increase in transformation efficiency to 19% and 18%, respectively. Lipoic acid also reduced the percentage of escapes from 50% in the control to 16% and 21% for 8 μM and 9 μM, respectively.

Media Compositions:

MSO—4.4 g/L MS salts

2 mL/L Gamborg's B5 salts

30 g/L Sucrose

Z Media—

Basal Formula—4.4 g/L MS salts

50 mL/L Nitsch 100× vitamins

0.5 mg/L Folic Acid

30 g/L Sucrose

9.0 g/L Washed Agar

Selection Media—All of the above components plus the following additives:

Z1 Nonselection

750 mg/L carbenicillin

100 mg/L cefotaxime

0.1 mg/L naphthaleneacetic acid

5 mg/L zeatin riboside

0.6-0.9 mg/L lipoic acid (depending upon concentration desired)

10 mg/L silver nitrate

Z1 Glyphosate Selection

750 mg/L carbenicillin

100 mg/L cefotaxime

0.1 mg naphthaleneacetic acid

5 mg/L zeatin riboside

5 mL/L glyphosate (0.005 mM)

0.6-0.9 mg/L lipoic acid (depending upon concentration desired)

10 mg/L silver nitrate

Z2 Glyphosate Selection

750 mg/L carbenicillin

100 mg/L cefotaxime

0.3 mg/L gibberellic acid

5 mg/L zeatin riboside

5 mL/L glyphosate (0.005 mM)

0.6-0.9 mg/L lipoic acid (depending upon concentration desired)

10 mg/L silver nitrate

### Example 3

### Wheat Transformation

This example describes the use of lipoic acid in an Agrobacterium-based wheat transformation process. Immature embryos of wheat (Triticum aestivum L) cv Bobwhite were isolated from the immature caryopsis (wheat spikelets) 13-15 days after pollination, and cultured on M7 media (M7 medium is based on CM4C medium [Table 8], but the amount of MS salts was doubled and the picloram concentration was increased from 2.4 mg/L to 4 mg/L) containing 0.02 mM glyphosate for 3-4 days in the dark at 25° C. The embryos without embryogenic callus were selected for Agrobacterium inoculation.

TABLE 8
Supplemental Components in Basal Media¹

| Components | CM4 | CM4C | MMSO.2C | MMSO |
|---|---|---|---|---|
| 2,4-D (mg/L) | 0.5 | 0.5 | 0.2 | — |
| Picloram (mg/L)² | 2.2 | 2.2 | | |
| Maltose (g/L) | 40.0 | 40.0 | 40.0 | 40.0 |
| Glutamine (g/L) | 0.5 | 0.5 | | |
| Magnesium Chloride (g/L) | 0.75 | 0.7 | | |
| Casein Hydrolysate (g/L) | | 0.1 | 0.1 | |
| MES (g/L) | | 1.95 | 1.95 | 1.95 |
| Ascorbic Acid (mg/L)² | | 100.0 | 100.0 | 100.0 |
| Gelling Agent (g/L)³ | 2(P) | 2(P) | 2(G) | 2(G) |

¹All media contain basal salts (MS basal salts) and vitamins (MS vitamins) from Murashige and Skoog (1962). The pH in each medium was adjusted to 5.8.
²Filter-sterilized and added to the medium after autoclaving.
³PHYTAGEL (P) (a registered trademark of Sigma Chemical Co., St. Louis, MO) or GELRITE (G) (available from Schweizerhall, Inc., South Plainfield NJ; a registered trademark of Monsanto Company, St. Louis, MO).

After preculture, 100 embryos were inoculated in 5 mL 1/10 CM4C medium with 5 mL Agrobacterium (OD₆₆₀=1), 10 μL Pluronic F68 was added, and the mixture was incubated for 15 minutes in the dark at room temperature. Embryos were placed on sterilized filter paper with 200 μL deionized water and cocultured in the dark for 3 days at 23° C. Embryos were then transferred to delay media for 7 days in the dark at 25° C. Delay medium consists of CM4C, 500 mg/L carbenicillin, and 0.02 mM glyphosate. Selection was then performed on CM4C with 2 mM glyphosate, 500 mg/L carbenicillin and aromatic amino acids (10⁻⁷/amino acid) for 7 days. Regeneration was started on MMSO.2C with 0.1 mM glyphosate, 250 mg/L carbenicillin, and aromatic amino acids (107/amino acid) for 2 weeks in the light at 25° C. Regeneration was continued in MMSO.2C with 0.02 mM glyphosate, 500 mg/L carbenicillin, aromatic amino acids (10⁻⁷/amino acid), and 2 μM copper sulfate for 2-8 weeks (with a transfer every 2 weeks) in the light at 25° C. Finally, plantlets were transferred to sundae cups with MMSO media with 0.02 mM glyphosate, and 500 mg/L carbenicillin.

TABLE 9
Different concentrations of lipoic acid in delay,
selection and first regeneration media.

| Concentration | | Responding calli | Efficiency |
|---|---|---|---|
| (μM) | Explants | (%) | (%) |
| 0 | 470 | 32.5 | 3.0 |
| 5 | 472 | 33.1 | 3.8 |
| 10 | 486 | 38.8 | 3.2 |
| 30 | 486 | 38.6 | 3.0 |
| 50 | 482 | 47.9 | 5.1 |
| 100 | 464 | 28.4 | 2.2 |

TABLE 10
Effect of lipoic acid at different stages
of selection and regeneration.

| Delay-selection-first | | | |
|---|---|---|---|
| regeneration | Explants | % responding calli | % efficiency |
| 0-0-0 | 582 | 40.8 | 2.9 |
| 25-25-50 (μM) | 599 | 41.7 | 3.0 |
| 25-50-50 (μM) | 560 | 50.5 | 5.4 |
| 50-50-50 (μM) | 570 | 49.6 | 4.9 |

Table 9 shows that 50 μM of lipoic acid in the delay, selection, and regeneration media increases the percentage of responding calli from 32.5% to 47.9% and increases the efficiency from 3.0% to 5.1%. Of the concentrations tested, 50 μM was the only effective concentration. When different concentrations were tried in the delay, selection and first regeneration media (Table 10), 25 μM in the delay medium, 50 μM in the selection medium, and 50 μM in the first regeneration medium also were effective, giving 5.4% transformation efficiency compared with 2.9% in the control. The results for 50 μM in each medium corresponds with the previous experiment, giving 5.4% transformation efficiency.

### Example 4

### Soybean Transformation

Dry A3244 soybean seeds were germinated by soaking in sterile distilled water (SDW) for three minutes, drained and allowed to slowly imbibe for 2 hours at which time Bean Germination Media (BGM) was added. At approximately 12 hours, seed axis explants were isolated by removing seed coats and cotyledons. Inoculation occurred 14 hours after the addition of SDW.

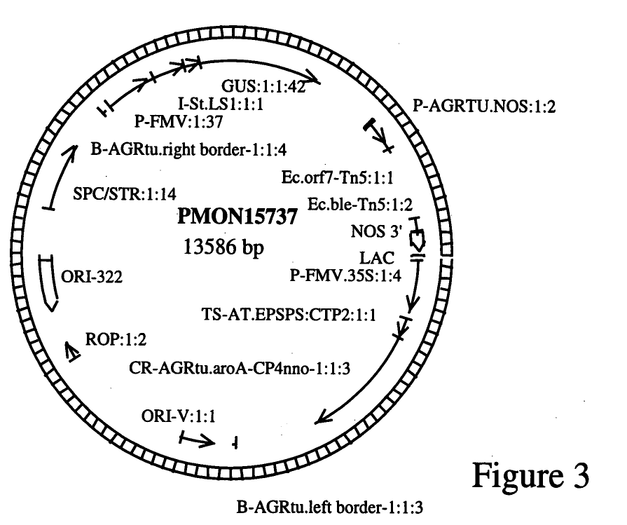

Explants were placed into sterile Plantcons™ with 20 mL of pMON15737/ABI (FIG. 3) and resuspended to an optical density A₆₆₀ of approximately 0.3 in 1/10 Gamborg's B5 media (Gamborg et al., Exp. Cell Res., 50:151-158, 1968) containing 3% glucose, 1.68 mg/L BAP, 3.9 g/L MES, 0.2M acetosyringone, 1 mM galactronic acid, and 0.25 mg/L GA₃. Each Plantcon™ was sonicated for 20 seconds in a L&R Quantrex S140 sonicator that contained SDW+0.1% Triton X100 in the bath. Plantcons™ were held in place at approximately 2.5 cm below the surface of the bath liquid. Following sonication, explants were inoculated for an additional hour while shaking gently on an orbital shaker at ˜90 RPM. After inoculation, the Agrobacterium was removed. One sheet of square filter paper and 3 mL of co-culture media containing 0-500 μM lipoic acid were added. Co-culture media consisted of 1/10 Gamborg's B5 media containing 5% glucose, 1.68 mg/L BAP, 3.9 g/L, 0.2M acetosyringone, 1 mM galactronic acid and 0.25 mg/L GA₃. Explants were incubated at 23° C. in the dark for 3 days.

Lipoic acid was dissolved in either ethanol or potassium hydroxide to make 100 mg/mL stock. Lipoic acid stock was added to co-culture media to final concentrations of 0, 10, 50, 100, 250, 500 μM lipoic acid.

Following co-culture, explants were transferred to solid Woody Plant Media (WPM) (McCown & Lloyd, Proc. International Plant Propagation Soc., 30:421, 1981) containing 2% sucrose, 200 mg/L carbenicillin, 100 mg/L Timetin (ticarcillin+clavulanic acid), +/−100 mg/L cefotaxime, 60 mg/L Benomyl, and 75 μM glyphosate. All explants were placed at 28° C., 16-hour light/8-hour dark photoperiod. A second transfer to the same media was made after two weeks.

Shoots were cut 5-8 weeks post-inoculation and rooted on Bean Rooting Media (BRM) containing 25 μM glyphosate and 100 mg/L Timetin.

As shown in Tables 11 and 12, low levels of lipoic acid (5 to 100 μM) were not particularly effective with the soybean transformation protocol used in this Example, but lipoic acid concentrations of 250 μM to 1500 μM provided increased transformation efficiency and increased shoot and rooted plant production. A lipoic acid concentration of 250 μM increased transformation frequency about ninefold. No significant consistent difference was observed whether the lipoic acid was dissolved in ethanol or potassium hydroxide.

TABLE 11
Effect of lipoic acid in soybean with effects
of dissolving in ethanol or potassium hydroxide.

| | | | | | Trans- |
|---|---|---|---|---|---|
| | Total | Total | Shooting | Rooted | formation |
| Treatment | Explants | Shoots | Frequency | Plants | Efficiency |
| Control, 0 uM | 472 | 25 | 5% | 4 | 0.8% |
| 0.05% ETOH | 478 | 34 | 7% | 10 | 2.1% |
| 250 uM LA | 486 | 69 | 14% | 7 | 1.4% |
| 250 uM LA, | 505 | 63 | 12% | 13 | 2.6% |
| KOH | | | | | |
| 0.10% ETOH | 556 | 44 | 8% | 9 | 1.6% |
| 500 uM LA | 531 | 64 | 12% | 9 | 1.7% |
| 500 uM LA, | 462 | 44 | 10% | 7 | 1.5% |
| KOH | | | | | |
| 0.15% ETOH | 551 | 33 | 6% | 3 | 0.5% |
| 750 uM LA | 535 | 62 | 12% | 9 | 1.7% |
| 750 uM LA, | 454 | 56 | 12% | 7 | 1.5% |
| KOH | | | | | |
| 0.20% ETOH | 485 | 30 | 6% | 6 | 1.2% |
| 1000 uM LA | 530 | 52 | 10% | 12 | 2.3% |
| 1000 uM LA, | 471 | 40 | 8% | 6 | 1.3% |
| KOH | | | | | |
| 0.30% ETOH | 483 | 30 | 6% | 3 | 0.6% |
| 1500 uM LA | 538 | 53 | 10% | 13 | 2.4% |
| 1500 uM LA, | 498 | 57 | 11% | 10 | 2.0% |
| KOH | | | | | |

TABLE 12
Effect of lower levels of lipoic acid on soybean.

| | | | | | Trans- |
|---|---|---|---|---|---|
| | Total | | | Total | formation |
| | Ex- | Total | Shooting | Rooted | Effic- |
| Treatment | plants | Shoots | Frequency | Plants | iency |
| Control, 0 uM | 490 | 37 | 8% | 3 | 0.6% |
| 5 uM LA | 244 | 18 | 7% | 3 | 1.2% |
| 5 uM LA, KOH | 248 | 13 | 5% | 2 | 0.8% |
| 10 uM LA | 247 | 14 | 6% | 0 | 0.0% |
| 10 uM LA, KOH | 240 | 13 | 5% | 0 | 0.0% |
| 50 uM LA | 242 | 22 | 9% | 1 | 0.4% |
| 50 uM LA, KOH | 246 | 17 | 7% | 1 | 0.4% |
| 100 uM LA | 396 | 39 | 10% | 3 | 0.8% |
| 100 uM LA, KOH | 246 | 18 | 7% | 2 | 0.8% |
| 250 uM LA | 246 | 32 | 13% | 9 | 3.7% |
| 500 uM LA* | 171 | 19 | 11% | 5 | 2.9% |

Bean Germination Media (BGM 2.5%)

| COMPOUND: | QUANTITY PER LITER | |
|---|---|---|
| BT STOCK #1 | 10 | mL |
| BT STOCK #2 | 10 | mL |
| BT STOCK #3 | 3 | mL |
| BT STOCK #4 | 3 | mL |
| BT STOCK #5 | 1 | mL |
| SUCROSE | 25 | g |

Adjust to pH 5.8.

Dispensed in 1 Liter Media Bottles, Autoclaved

| ADDITIONS PRIOR TO USE: | PER 1 L |
|---|---|
| CEFOTAXIME (50 mg/mL) | 2.5 mL |
| FUNGICIDE STOCK | 3 mL |

Bt Stock for Bean Germination Medium (BGM)

Make and store each stock individually. Dissolve each chemical thoroughly in the order listed before adding the next. Adjust volume of each stock accordingly. Store at 4° C.

Bt Stock 1 (1 liter)

| | | |
|---|---|---|
| KNO₃ | 50.5 | g |
| NH₄NO₃ | 24.0 | g |
| MgSO₄*7H₂O | 49.3 | g |
| KH₂PO₄ | 2.7 | g |

Bt Stock 2 (1 liter)

| | | |
|---|---|---|
| CaCl₂*2H₂O | 17.6 | g |

Bt Stock 3 (1 liter)

| | | |
|---|---|---|
| H₃BO₃ | 0.62 | g |
| MnSO₄-H₂O | 1.69 | g |
| ZnSO₄-7H₂O | 0.86 | g |
| KI | 0.083 | g |
| NaMoO₄-2H₂O | 0.072 | g |
| CuSO₄-5H₂O | 0.25 | mL of 1.0 |
| | | mg/mL stock |
| CoCl₄-6H₂O | 0.25 | mL of 1.0 |
| | | mg/mL stock |

Bt Stock 4 (1 liter)

| | | |
|---|---|---|
| Na₂EDTA | 1.116 | g |
| FeSO₄7H₂O | 0.834 | g |

Bt Stock 5 (500 mL) Store in a foil wrapped container

| | | |
|---|---|---|
| Thiamine-HCl | 0.67 | g |
| Nicotinic Acid | 0.25 | g |
| Pyridoxine-HCl | 0.41 | g |

FUNGICIDE STOCK (100 mL)

| | | |
|---|---|---|
| chlorothalonile (75% WP) | 1.0 | g |
| benomyl (50% WP) | 1.0 | g |
| captan (50% WP) | 1.0 | g |
| Add to 100 mL of sterile distilled water. | | |
| Shake well before using. | | |
| Store 4° C. dark for no more than one week. | | |

BEAN ROOTING MEDIA (BRM) (for 4L)

| MS Salts | 8.6 | g |
|---|---|---|
| Myo-Inositol (Cell Culture Grade) | .40 | g |
| Soybean Rooting Media Vitamin Stock | 8 | mL |
| L-Cysteine (10 mg/mL) | 40 | mL |
| Sucrose (Ultra Pure) | 120 | g |
| | pH 5.8 | |
| Washed Agar | 32 | g |

ADDITIONS AFTER AUTOCLAVING:

| | | |
|---|---|---|
| BRM Hormone Stock | 20.0 | mL |
| Ticarcillin/clavulanic acid (100 mg/mL Ticarcillin) | 4.0 | mL |

VITAMIN STOCK FOR SOYBEAN ROOTING MEDIA (1 liter)

| | | |
|---|---|---|
| Glycine | 1.0 | g |
| Nicotinic Acid | 0.25 | g |
| Pyridoxine HCl | 0.25 | g |
| Thiamine HCl | 0.05 | g |

Dissolve one ingredient at a time, bring to volume, store in foil-covered bottle in refrigerator for no more than one month.

BRM Hormone Stock

Amount for 1 Liter

6.0 mL IAA (0.033 mg/mL)

4.0 mL SDW

Store dark at 4° C.

### Example 5

### Cotton Transformation

Cotton seed was washed in 2 teaspoons of detergent in 700-800 mL of sterile water for 10 minutes. Seed was then soaked in fungicide solution (0.6 g benlate, 0.6 g captan per 100 mL sterile water) for 10 min, drained, but not rinsed. Seed was imbibed for 30 minutes in 800 mL of 50% Clorox with 3 drops of Tween-20 and then rinsed 3 times with sterile water. Seed was spread in a sterile phytatray containing 1/2 MS plus 1% glucose media at a density of approximately 20 seeds per tray. Seeds were germinated in the dark at 28° C. for 7-10 days.

Agrobacterium was prepared by streaking a solid LBssck plate from frozen stock and incubating at 28° C. for 2 days. Two mL of liquid LBssck was inoculated with a single colony from the plate and grown overnight at 28° C. Culture is diluted 1:10 with fresh LBssck and cultured overnight at 28° C. The cells were spun down, the supernatant was poured off, cells were washed with MSO media and then resuspended in 20 mL of MSO.

Explants were harvested by cutting off the cotyledons and then making a cut approximately 4 to 6 cm below the cotyledon. Hypocotyl sections were placed in MSO media and then cut into 0.5 to 1 cm pieces.

Twenty mL of Agrobacterium broth was added to 100 to 500 cut explant sections and allowed to soak for 20 minutes. Explants were moved to co-culture medium (TrCO; see media descriptions below), covered with a plastic bag and co-cultured at room temperature for 2 days. After co-culture, explants were moved to UMSEL (1) media (with kanamycin or glyphosate selection), parafilmed and cultured in the light (16/8 photoperiod) at 28° C. for 4 weeks. Tissue was then transferred to fresh UMSEL (2) media containing selection in the same culture conditions for an additional 4 weeks.

Independent calli that had formed were then moved to UMO(1) containing selection, parafilmed and cultured in the dark at 28° C. for 8 weeks. After 8 weeks, tissue was transferred to fresh UMO(2) containing selection, parafilmed, and cultured in the dark for an additional 8 weeks. Good embryogenic callus was moved to TRP embryo maturation media, parafilmed and cultured in the dark at 28° C. Embryogenic callus was subcultured to fresh TRP media every 4 weeks until somatic embryos formed and were moved to SHSU embryo germination media. Once on parafilmed SHSU plates, the shoots were placed in the light at 28° C. and subcultured every four weeks until roots began to form. When healthy roots and leaves were present, the shoots were moved to SHSU media in sundae cups and eventually to soil.

TABLE 13
Effect of lipoic acid for 8 weeks in UMSEL
media (average from 3 experiments).

| | Putative | |
|---|---|---|
| Lipoic acid (μM) | embryogenic calli | Frequency (%) |
| 0 | 121 | 41.4 |
| 5 | 132 | 49.4 |
| 10 | 88 | 31.4 |
| 50 | 120 | 61.2 |
| 100 | 107 | 56.6 |

As shown in Table 13, the inclusion of lipoic acid at a concentration of 50 μM or 100 μM in the selection stage of cotton transformation and regeneration increased the frequency of embryogenic callus formation from 41.4% in the control to 61.2% and 56.6%, respectively.

Media Abbreviations (Numbers Based on 1 Liter of Media)

TRP=4.3 g MS salts, 2 mL B5 vitamins, 1.9 g KNO₃ (potassium nitrate), 30 g glucose, pH 5.8, 3.5 g Schweizer Hall. Plus 500 mg/L carbenicillin, 100 mg/L cefotaxime and 50 mg/L benlate.

TRP+=same as TRP (above) with the addition of 0.1 g casein hydrolysate.

TRCO=0.44 g MS salts, 2 mL B5 vitamins, 0.1 mg 2,4-D, 0.1 g kinetin, 30 g glucose, pH 5.8, 0.9% Sigma TC agar.

UMSEL=4.3 g MS salts, 2 mL B5 vitamins, 0.1 mL 2,4-D (1 mg/mL), 1 mL kinetin (0.5 mg/mL), 30 g glucose, pH 5.8, 2.5 g phytagel, 2 mL (250 mg/mL) carbenicillin, 1 mL (100 mg/mL) cefotaxime, plus selection.

UMO=4.3 g MS salts, 2 mL B5 vitamins, 30 g glucose, pH 5.8, 3.5 g phytagel, 2 mL (250 mg/mL) carbenicillin, 1 mL (100 mg/mL) cefotaxime, 100 mg/L ascorbic acid, plus selection.

SHSU=100 mL Stewart & Hsu Majors (10×), 10 ml Stewart & Hsu Minors (100×), 1.5 mL iron (100×), 10 mL Stewart & Hsu Organics (100×), 5 g glucose, 50 mg/L benlate, 2.2 g Schweizer Hall, pH 6.8. Reference for Stewart & Hsu media: Stewart, James M, & Cecilia L. Hsu; 1977; Planta, 137, 113-117.

MSO=4.4 g MS salts, 2 ml B5 vitamins, 30 g sucrose, pH 5.7.

LB=10 g NaCl (sodium chloride), 5 g yeast extract, 10 g tryptone (to make solid LB add 15 g agar), pH 7.0.

TXD=4.3 g MS salts, 2 mL B5 vitamins, 0.01 mL kinetin (0.5 mg/mL), 8 mL pCPA (0.5 mg/mL), 30 g sucrose, pH 5.7.

1/2 MS=2.5 g MS salts, 2 mL B5 vitamins, 2.5 g phytagel, pH 5.8.

LB/SSCK=regular LB media (see above), with the addition of 0.5 mL (100 mg/mL) spectinomycin, 0.5 mL (100 mg/mL) streptomycin, 1 mL (25 mg/mL) chloramphenicol, & 1 mL (50 mg/mL) kanamycin.

Abbreviations used in media formulations: TIBA=triiodobenzoic acid; CARB=carbenicillin; TIC=ticarcillin; KAN=kanamycin; VANCO=vancomycin; CEF=cefotaxime; RIF=rifampicin; TIME=timetin; pCPA=p-Chlorophenoxyacetic acid; STREP=streptomycin; CHLORAM=chloramphenicol; SPEC=spectinomycin.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the invention has been described in detail for the purpose of illustration, it is understood that such detail is solely for that purpose, and variations can be made therein by those skilled in the art without departing from the spirit and scope of the invention which is defined by the following claims.

What is claimed is:

1. A method for introducing a nucleic acid sequence into the genome of a plant cell and regenerating a transformed plant therefrom, said method comprising culturing said plant cell on at least one plant transformation media, said at least one plant transformation media comprising an effective amount of lipoic acid or an analog thereof.

2. The method of claim 1 wherein the amount of lipoic acid or an analog thereof in said plant transformation media is between about 2 μM and about 2000 μM.

3. The method of claim 1 wherein the amount of lipoic acid or an analog thereof in said plant transformation media is between about 5 μM and about 1500 μM.

4. The method of claim 1 wherein the amount of lipoic acid or an analog thereof in said plant transformation media is between about 5 μM and about 100 μM.

5. A plant transformation media comprising an effective amount of lipoic acid or an analog thereof.

6. The media of claim 5 wherein the amount of lipoic acid or an analog thereof in said plant transformation media is between about 2 μM and about 2000 μM.

7. The media of claim 5 wherein the amount of lipoic acid or an analog thereof in said plant transformation media is between about 5 μM and about 1500 μM.

8. The media of claim 5 wherein the amount of lipoic acid or an analog thereof in said plant transformation media is between about 5 μM and about 100 μM.

9. The media of claim 5 wherein said media is suitable for co-cultivation of plant cell or plant tissue with Agrobacterium.

10. The media of claim 5 wherein said media is suitable for the selection of transformed plant cells or tissues.

11. The media of claim 5 wherein said media is suitable for regeneration of transformed plant cells or tissues into whole fertile plants.

12. A method of producing a transformed tomato plant comprising
a) isolating a tomato explant suitable for transformation;
b) combining said tomato explant with a heterologous gene construct containing a gene of interest to produce a transformed tomato explant;
c) culturing said transformed tomato explant in a plant transformation media for selection and shoot induction to produce transformed shoots therefrom, said plant transformation media containing an effective amount of lipoic acid;
d) identifying said transformed shoots; and
e) rooting said transformed shoots to produce a transformed tomato plant.

13. A method of producing a transformed potato plant comprising
a) isolating a potato explant suitable for transformation;
b) combining said potato explant with a heterologous gene construct containing a gene of interest to produce a transformed potato explant;
c) culturing said transformed potato explant in a first plant transformation media containing an effective amount of lipoic acid until transformed shoots form from said explants;
d) rooting said transformed shoots to produce a transformed potato plant.

14. A method of producing a transformed wheat plant comprising
a) isolating a wheat explant suitable for transformation;
b) combining said wheat explant with a heterologous gene construct containing a gene of interest to produce a transformed wheat explant;
c) culturing said transformed wheat explant in a first plant transformation media containing an effective amount of lipoic acid and a selective agent to select for transformed wheat explants;
d) culturing said transformed wheat explants in a second plant transformation media containing an effective amount of lipoic acid to regenerate transformed shoots from said transformed wheat explants;
e) rooting said transformed shoots to produce a transformed wheat plant.

15. A method of producing a transformed soybean plant comprising
a) isolating a soybean explant suitable for transformation;
b) combining said soybean explant with a heterologous gene construct containing a gene of interest to produce a transformed soybean explant in a plant transformation media containing an effective amount of lipoic acid;
c) culturing said transformed soybean explant in a plant transformation media containing a selective agent to select for transformed soybean explants containing the gene of interest and producing transformed shoots therefrom; and
d) rooting said transformed shoots to produce a transformed soybean plant.
